(19) United States
(12) Patent Application Publication
(10) Pub. No.: US 2009/0306040 A1
(43) Pub. Date: Dec. 10, 2009

(54) Mono and Combination Therapy of M1/M4 Agonist (Sabcomeline) for Treatment of Negative Symptoms of Schizophrenia

(76) Inventors: Paul Christopher Sharpe, Essex (GB); Peter Robin Blower, Essex (GB); Jill Galloway Chisnall Rasmussen, Essex (GB)

Correspondence Address: NIXON & VANDERHYE, PC, 901 NORTH GLEBE ROAD, 11TH FLOOR, ARLINGTON, VA, 22203, US

(21) Appl. No.: 12/226,421

(22) PCT Filed: Apr. 23, 2007
(86) PCT No.: PCT/GB2007/001468

§ 371 Date: Jan. 16, 2009

(30) Foreign Application Priority Data
Apr. 21, 2006 (GB) 0607949.5

(51) Int. Cl. A61K 31/397; A61K 31/20; A61K 31/54; A61P 25/18

(52) U.S. Cl. 514/210.15; 514/560; 514/227.5

(57) ABSTRACT

The invention relates to the use of a functional muscarinic M1/M4 receptor agonist or a pharmaceutically acceptable salt thereof for the treatment of negative symptoms of schizophrenia. It also relates to adjunctive and simultaneous combination therapies for the treatment of negative symptoms of schizophrenia in which the functional muscarinic M1/M4 receptor agonist or a pharmaceutically acceptable salt thereof, in particular sabcomeline or a pharmaceutically acceptable salt thereof and at least one neuroprotective agent, neuroleptic and/or atypical antipsychotic agent are administered adjunctively or simultaneously. The invention also provides methods of treatment of the negative symptoms of schizophrenia utilising such therapies and such adjunctive or simultaneous therapeutic combination therapies, therapeutic combinations for use therein and pharmaceutical compositions comprising them.

The invention relates to the treatment of negative symptoms of schizophrenia, to therapeutic combinations and combinations comprising them for use in the treatment of negative symptoms of schizophrenia, and to methods of treatment of negative symptoms of schizophrenia.

U.S. Pat. No. 5,278,170 describes a class of compounds which enhance cholinergic neuronal activity via functional action at muscarinic M1/M4 receptors within the central nervous system. A particularly preferred compound from within the scope of this disclosure has been given the common name sabcomeline. The chemical name for sabcomeline is R-(Z)-α-(methoxyimino)-α-(1-azabicyclo[2.2.2]oct-3-yl)acetonitrile. For therapeutic administration, it is preferably used in the form of a pharmaceutically acceptable salt, typically the hydrochloride salt, but alternative salts of sabcomeline with pharmaceutically acceptable acids may also be utilised in therapeutic administration, for example salts derived from sabcomeline free base and acids including, but not limited to, hydrobromic acid, phosphoric acid, acetic acid, fumaric acid, maleic acid, salicylic acid, citric acid, lactic acid, oxalic acid and p-toluene sulphonic acid. Sabcomeline was initially evaluated for its use in the treatment of Alzheimer's disease.

WO 02/03684 discloses the treatment of psychotic disorders including schizophrenia and mania by administration of a muscarinic receptor agonist in combination with a typical or an atypical antipsychotic agent. As referred to below, negative symptoms associated with schizophrenia appear to be responsible for much of the chronic and long-term disability associated with the disorder. Although treatment is available for schizophrenia, including treatment with a large number of typical and atypical antipsychotic agents, optionally in combination with muscarinic receptor agonists, current treatments for schizophrenia have shown limited benefit in the treatment of negative symptoms. There is no disclosure in WO 02/03684 of any activity of any muscarinic receptor agonist combinations against negative symptoms of schizophrenia, nor is there any enabling disclosure of how to demonstrate or optimise such activity.

Symptoms of schizophrenia are divided into two broad classes: positive symptoms and negative symptoms.

Positive symptoms generally involve the experience of something in consciousness that should not normally be present. For example, hallucinations and delusions represent perceptions or beliefs that should not normally be experienced.

In addition to hallucinations and delusions, patients with schizophrenia frequently have marked disturbances in the logical process of their thoughts. Specifically, psychotic thought processes are characteristically loose, disorganised, illogical, or bizarre. These disturbances in thought process frequently produce observable patterns of behaviour that are also disorganised and bizarre. The severe disturbances of thought content and process that comprise the positive symptoms often are the most recognisable and striking features of schizophrenia.

Further positive symptoms of schizophrenia include, for example, disorganised thoughts and behaviours, loose or illogical thoughts, agitation, and disorganised speech including loose associations (derailment), incoherence and blocking.

One of the commonest of the positive symptoms that are associated from schizophrenia is hallucinations. Hallucinations are said to occur when an individual experiences a sensory impression that has no basis in reality. This impression could involve any of the sensory modalities. Thus hallucinations may be auditory, olfactory, gustatory, kinesthetic, tactile or visual. For example, auditory hallucinations frequently involve the impression that one is hearing a voice. In each case, the sensory impression is falsely experienced as real.

Another manifestation of positive symptoms resulting from schizophrenia is delusions. A delusion is a false belief that an individual holds despite evidence to the contrary. A common example is paranoia, in which a person has delusional beliefs that others are trying to harm him or her. Attempts to persuade the person that these beliefs are unfounded typically fail and may even result in the further entrenchment of the beliefs. Hallucinations and delusions are among the most commonly observed positive symptoms of schizophrenia and are the basis for all schizophrenia treatments available currently.

In addition to positive symptoms, patients with schizophrenia have been noted to exhibit major deficits in motivation and spontaneity and these are referred to as negative symptoms.

While positive symptoms represent the presence of something not normally experienced, negative symptoms reflect the absence of thoughts and behaviours that would otherwise be expected and thus reflect a decrease or loss of normal function or the loss or absence of normal behaviours. Negative symptoms of schizophrenia include, for example, flat or blunted affect, concrete thoughts, anhedonia (the inability to experience pleasure), poor motivation, spontaneity, and initiative. Inflexibility or rigidity of thought represents impairment in the ability to think abstractly. Blunting of affect refers to a general reduction in the ability to express emotion. Motivational failure and inability to initiate activities represent an important source of long-term disability in schizophrenia. Anhedonia reflects a deficit in the ability to experience pleasure and to react appropriately to pleasurable situations.

While positive symptoms such as hallucinations are responsible for much of the acute distress associated with schizophrenia, negative symptoms appear to be responsible for much of the chronic and long-term disability associated with the disorder. Current treatments for schizophrenia have shown limited benefit in the treatment of negative symptoms.

Negative symptoms of schizophrenia can be further subdivided into primary and secondary negative symptoms.

Primary negative symptoms do not include symptoms that are better accounted for by medication side-effects, post-psychotic depression or demoralization. Rather, examples of primary negative symptoms include: affective flattening (for example emotional immobility, unresponsiveness, poor eye contact, and limited body movement); alogia (this is where the patient exhibits poverty of speech and usually manifests itself by the patient making brief replies during conversation); avolition (the inability to initiate and persist in goal-directed activities); anhedonia (loss of interest or pleasure); dysphoric mood (depression, anxiety and anger); disturbances in sleep pattern (sleeping during the day, restlessness/night-time activity); abnormal psychomotor activity (pacing, rocking, apathetic immobility); and lack of insight.

Secondary negative symptoms, some of which occur in association with positive symptoms and/or medication side-effects, include for example, movement disorders such as extrapyramidal symptoms, akathisia and tardive dyskinesia and demoralisation.

There remains a need to identify medicaments for use in the treatment of negative symptoms of schizophrenia, and in particular compositions and methods of treatment which improve on the efficacy of existing therapies.

It has now been found that functional muscarinic M1/M4 receptor agonists such as sabcomeline or a pharmaceutically acceptable salt thereof may advantageously be administered to treat negative symptoms of schizophrenia.

For the avoidance of doubt, it is intended that the term schizophrenia covers the full spectrum of schizophrenic disorders known to the skilled person. These include, but are not limited to, the following: catatonic, disorganised, paranoid, residual and undifferentiated schizophrenia; schizophreniform disorder and schizoaffective disorder.

In a first aspect therefore, the invention provides a method of treatment of negative symptoms of schizophrenia by administration of a functional muscarinic M1/M4 receptor agonist. In a further aspect, the invention provides the use of a functional muscarinic M1/M4 receptor agonist in the manufacture of a medicament for the treatment of negative symptoms of schizophrenia. The invention also provides the use of functional muscarinic M1/M4 receptor agonist for the treatment of negative symptoms of schizophrenia. The invention further provides functional muscarinic M1/M4 receptor agonist for use in the treatment of negative symptoms of schizophrenia.

Functional muscarinic M1/M4 receptor agonists are compounds which enhance cholinergic neuronal activity at the muscarinic M1/M4 receptors predominantly. This functional selectivity results in a level of safety and tolerability advantageous for use in the treatment of negative symptoms of schizophrenia. Sabcomeline is one such functional muscarinic M1/M4 receptor agonist. Other suitable functional M1/M4 receptor agonists or combinations thereof may also be used.

For therapeutic administration according to the present invention, the functional muscarinic M1/M4 receptor agonist, in particular sabcomeline may be employed in the form of its free base, but is preferably used in the form of a pharmaceutically acceptable salt, typically the hydrochloride salt.

Alternative salts of the functional muscarinic M1/M4 receptor agonist, in particular sabcomeline with pharmaceutically acceptable acids may also be utilised in therapeutic administration, for example salts derived from the functional muscarinic M1/M4 receptor agonist, in particular sabcomeline free base and acids including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid.

All solvates and all alternative physical forms of the functional muscarinic M1/M4 receptor agonist, in particular sabcomeline or its pharmaceutically acceptable derivatives as described herein, including but not limited to alternative crystalline forms, amorphous forms and polymorphs are also within the scope of this invention, and all references to the functional muscarinic M1/M4 receptor agonist, in particular sabcomeline herein include all pharmaceutically acceptable salts, and all solvates and alternative physical forms thereof.

For therapeutic administration according to the invention, the functional muscarinic M1/M4 receptor agonist or a pharmaceutically acceptable salt thereof, in particular sabcomeline or its pharmaceutically acceptable salts or solvates may be administered in pure form, but will preferably be formulated into any suitable pharmaceutically acceptable and effective composition which provides effective levels of the active ingredient in the body.

The treatment of negative symptoms of schizophrenia may include administering the functional muscarinic M1/M4 receptor agonist or a pharmaceutically acceptable salt thereof, in particular sabcomeline or a pharmaceutically acceptable salt thereof at a dose of between 10 μg-200 μg. Preferably, the dose is between 20 μg-100 μg. More preferably, the dose is between 25 μg-50 μg. The dose may be administered as a single dose or twice daily. Ideally, the functional muscarinic M1/M4 receptor agonist or a pharmaceutically acceptable salt thereof, in particular sabcomeline or a pharmaceutically acceptable salt thereof is administered at a dose of 25 μg twice daily.

Typically, the functional muscarinic M1/M4 receptor agonist or a pharmaceutically acceptable salt thereof, in particular sabcomeline or a pharmaceutically acceptable salt thereof is administered to the patient at dose ranges of 20 to 50 μg total daily dose with titration to optimal dose in the range 10 to 200 μg total daily dose.

Less preferably, the functional muscarinic M1/M4 receptor agonist or a pharmaceutically acceptable salt thereof, in particular sabcomeline or a pharmaceutically acceptable salt thereof is administered independently of any other medication.

It has also been found that the functional muscarinic M1/M4 receptor agonist or a pharmaceutically acceptable salt thereof, in particular sabcomeline or a pharmaceutically acceptable salt thereof may advantageously be administered in combination with at least one neuroprotective agent and/or neuroleptic agent (which may be a typical or atypical antipsychotic agent) to provide improved treatment of negative symptoms of schizophrenia. The combinations, uses and methods of treatment of the invention may also provide advantages in treatment of patients who fail to respond adequately or who are resistant to other known treatments.

In a further aspect, the invention provides a method of treatment of the negative symptoms of schizophrenia by adjunctive therapeutic administration of the functional muscarinic M1/M4 receptor agonist or a pharmaceutically acceptable salt thereof, in particular sabcomeline or a pharmaceutically acceptable salt thereof to a patient receiving therapeutic administration of at least one neuroprotective agent and/or neuroleptic agent (which may be a typical or atypical antipsychotic agent).

In a further aspect, the invention provides a method of treatment of the negative symptoms of schizophrenia by adjunctive therapeutic administration of the functional muscarinic M1/M4 receptor agonist or a pharmaceutically acceptable salt thereof, in particular sabcomeline or a pharmaceutically acceptable salt thereof to a patient receiving therapeutic administration of at least one neuroprotective agent.

In a further aspect, the invention provides a method of treatment of the negative symptoms of schizophrenia by adjunctive therapeutic administration of the functional muscarinic M1/M4 receptor agonist or a pharmaceutically acceptable salt thereof, in particular sabcomeline or a pharmaceutically acceptable salt thereof to a patient receiving therapeutic administration of at least one neuroleptic agent.

In a further aspect, the invention provides a method of treatment of the negative symptoms of schizophrenia by adjunctive therapeutic administration of the functional muscarinic M1/M4 receptor agonist or a pharmaceutically acceptable salt thereof, in particular sabcomeline or a pharmaceutically acceptable salt thereof to a patient receiving therapeutic administration of at least one typical antipsychotic agent.

In a further aspect, the invention provides a method of treatment of the negative symptoms of schizophrenia by adjunctive therapeutic administration of the functional muscarinic M1/M4 receptor agonist or a pharmaceutically acceptable salt thereof, in particular sabcomeline or a pharmaceutically acceptable salt thereof to a patient receiving therapeutic administration of at least one atypical antipsychotic agent.

In a further aspect, the invention provides the use of the functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of negative symptoms of schizophrenia in a patient receiving therapeutic administration of at least one neuroprotective and neuroleptic agent (which may be a typical or atypical antipsychotic agent).

In a further aspect, the invention provides the use of the functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of negative symptoms of schizophrenia in a patient receiving therapeutic administration of at least one neuroprotective agent.

In a further aspect, the invention provides the use of the functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of negative symptoms of schizophrenia in a patient receiving therapeutic administration of at least one neuroleptic agent.

In a further aspect, the invention provides the use of the functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of negative symptoms of schizophrenia in a patient receiving therapeutic administration of at least one typical antipsychotic agent.

In a further aspect, the invention provides the use of the functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of negative symptoms of schizophrenia in a patient receiving therapeutic administration of at least one atypical antipsychotic agent.

The invention also provides the use of the functional muscarinic M1/M4 receptor agonist or a pharmaceutically acceptable salt thereof, in particular sabcomeline or a pharmaceutically acceptable salt thereof in adjunctive therapeutic administration for the treatment of negative symptoms of schizophrenia in a patient receiving therapeutic administration of at least one neuroprotective agent and/or neuroleptic agent (which may be a typical or atypical antipsychotic agent).

The invention also provides the use of the functional muscarinic M1/M4 receptor agonist or a pharmaceutically acceptable salt thereof, in particular sabcomeline or a pharmaceutically acceptable salt thereof in adjunctive therapeutic administration for the treatment of negative symptoms of schizophrenia in a patient receiving therapeutic administration of at least one neuroprotective agent.

The invention also provides the use of the functional muscarinic M1/M4 receptor agonist or a pharmaceutically acceptable salt thereof, in particular sabcomeline or a pharmaceutically acceptable salt thereof in adjunctive therapeutic administration for the treatment of negative symptoms of schizophrenia in a patient receiving therapeutic administration of at least one neuroleptic agent.

The invention also provides the use of the functional muscarinic M1/M4 receptor agonist or a pharmaceutically acceptable salt thereof, in particular sabcomeline or a pharmaceutically acceptable salt thereof in adjunctive therapeutic administration for the treatment of negative symptoms of schizophrenia in a patient receiving therapeutic administration of at least one typical antipsychotic agent.

The invention also provides the use of the functional muscarinic M1/M4 receptor agonist or a pharmaceutically acceptable salt thereof, in particular sabcomeline or a pharmaceutically acceptable salt thereof in adjunctive therapeutic administration for the treatment of negative symptoms of schizophrenia in a patient receiving therapeutic administration of at least one atypical antipsychotic agent.

The invention also provides the functional muscarinic M1/M4 receptor agonist or a pharmaceutically acceptable salt thereof, in particular sabcomeline or a pharmaceutically acceptable salt thereof for use for adjunctive therapeutic administration for the treatment of negative symptoms of schizophrenia in a patient receiving therapeutic administration of at least one neuroprotective agent and/or neuroleptic agent (which may be a typical or atypical antipsychotic agent).

The invention also provides the functional muscarinic M1/M4 receptor agonist or a pharmaceutically acceptable salt thereof, in particular sabcomeline or a pharmaceutically acceptable salt thereof for use for adjunctive therapeutic administration for the treatment of negative symptoms of schizophrenia in a patient receiving therapeutic administration of at least one neuroprotective agent.

The invention also provides the functional muscarinic M1/M4 receptor agonist or a pharmaceutically acceptable salt thereof, in particular sabcomeline or a pharmaceutically acceptable salt thereof for use for adjunctive therapeutic administration for the treatment of negative symptoms of schizophrenia in a patient receiving therapeutic administration of at least one neuroleptic agent.

The invention also provides the functional muscarinic M1/M4 receptor agonist or a pharmaceutically acceptable salt thereof, in particular sabcomeline or a pharmaceutically acceptable salt thereof for use for adjunctive therapeutic administration for the treatment of negative symptoms of schizophrenia in a patient receiving therapeutic administration of at least one typical antipsychotic agent.

The invention also provides the functional muscarinic M1/M4 receptor agonist or a pharmaceutically acceptable salt thereof, in particular sabcomeline or a pharmaceutically acceptable salt thereof for use for adjunctive therapeutic administration for the treatment of negative symptoms of schizophrenia in a patient receiving therapeutic administration of at least one atypical antipsychotic agent.

In a further aspect, the invention provides a method of treatment of the negative symptoms of schizophrenia by adjunctive therapeutic administration of at least one neuroprotective and/or neuroleptic agent (which may be a typical or atypical antipsychotic) to a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides a method of treatment of the negative symptoms of schizophrenia by adjunctive therapeutic administration of at least one neuroprotective agent to a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides a method of treatment of the negative symptoms of schizophrenia by adjunctive therapeutic administration of at least one neuroleptic agent to a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides a method of treatment of the negative symptoms of schizophrenia by adjunctive therapeutic administration of at least one typical antipsychotic agent to a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides a method of treatment of the negative symptoms of schizophrenia by adjunctive therapeutic administration of at least one atypical antipsychotic agent to a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides the use of at least one neuroprotective and/or neuroleptic agent (which may be a typical or atypical antipsychotic agent) in the manufacture of a medicament for adjunctive therapeutic administration for the treatment of the negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides the use of at least one neuroprotective agent in the manufacture of a medicament for adjunctive therapeutic administration for the treatment of the negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides the use of at least one neuroleptic agent in the manufacture of a medicament for adjunctive therapeutic administration for the treatment of the negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides the use of at least one typical antipsychotic agent in the manufacture of a medicament for adjunctive therapeutic administration for the treatment of the negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides the use of at least one atypical antipsychotic agent in the manufacture of a medicament for adjunctive therapeutic administration for the treatment of the negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof. The invention also provides the use of at least one neuroprotective and/or neuroleptic agent (which may be a typical or atypical antipsychoticagent) for the treatment of negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

The invention also provides the use of at least one neuroprotective agent for the treatment of negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

The invention also provides the use of at least one neuroleptic agent for the treatment of negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

The invention also provides the use of at least one typical antipsychotic agent for the treatment of negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

The invention also provides the use of at least one atypical antipsychotic agent for the treatment of negative symptoms of schizophrenia in a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides a method of treatment of the negative symptoms of schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one neuroprotective agent and/or neuroleptic agent (which may be a typical or atypical antipsychotic agent).

In a further aspect, the invention provides a method of treatment of the negative symptoms of schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one neuroprotective agent.

In a further aspect, the invention provides a method of treatment of the negative symptoms of schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one neuroleptic agent.

In a further aspect, the invention provides a method of treatment of the negative symptoms of schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one typical antipsychotic agent.

In a further aspect, the invention provides a method of treatment of the negative symptoms of schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one atypical antipsychotic agent.

The invention further provides the use of a combination of the functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) in the manufacture of a medicament for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one neuroprotective agent in the manufacture of a medicament for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one neuroleptic agent in the manufacture of a medicament for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one typical antipsychotic agent in the manufacture of a medicament for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one atypical antipsychotic agent in the manufacture of a medicament for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one neuroprotective agent for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one neuroleptic agent for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one typical antipsychotic agent for simultaneous therapeutic administration in the treatment of the negative symptoms of schizophrenia.

The invention further provides the use of a combination of the functional muscarinic M1/M4 receptor agonist or a pharmaceutically acceptable salt thereof, in particular sabcomeline or a pharmaceutically acceptable salt thereof and at least one atypical antipsychotic agent for simultaneous therapeutic administration in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one neuroprotective agent and/or neuroleptic agent (which may be a typical or atypical antipsychotic agent) in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one neuroprotective agent in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one neuroleptic agent in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one typical antipsychotic agent in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one atypical antipsychotic agent in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof for simultaneous therapeutic administration with at least one neuroprotective agent and/or neuroleptic agent (which may be a typical or atypical antipsychotic agent) in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof for simultaneous therapeutic administration with at least one neuroprotective agent in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof for simultaneous therapeutic administration with at least one neuroleptic agent in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof for simultaneous therapeutic administration with at least one typical antipsychotic agent in the treatment of the negative symptoms of schizophrenia.

The invention further provides the use of the functional muscarinic M1/M4 receptor agonist or a pharmaceutically acceptable salt thereof, in particular sabcomeline or a pharmaceutically acceptable salt thereof for simultaneous therapeutic administration with at least one atypical antipsychotic agent in the treatment of the negative symptoms of schizophrenia.

The invention further provides the functional muscarinic M1/M4 receptor agonist or a pharmaceutically acceptable salt thereof, in particular sabcomeline or a pharmaceutically acceptable salt thereof for use for simultaneous therapeutic administration with at least one neuroprotective agent and/or neuroleptic agent (which may be a typical or atypical antipsychotic agent) in the treatment of the negative symptoms of schizophrenia.

The invention further provides the functional muscarinic M1/M4 receptor agonist or a pharmaceutically acceptable salt thereof, in particular sabcomeline or a pharmaceutically acceptable salt thereof for use for simultaneous therapeutic administration with at least one neuroprotective agent in the treatment of the negative symptoms of schizophrenia.

The invention further provides the functional muscarinic M1/M4 receptor agonist or a pharmaceutically acceptable salt thereof, in particular sabcomeline or a pharmaceutically acceptable salt thereof for use for simultaneous therapeutic administration with at least one neuroleptic agent in the treatment of the negative symptoms of schizophrenia.

The invention further provides the functional muscarinic M1/M4 receptor agonist or a pharmaceutically acceptable salt thereof, in particular sabcomeline or a pharmaceutically acceptable salt thereof for use for simultaneous therapeutic administration with at least one typical antipsychotic agent in the treatment of the negative symptoms of schizophrenia.

The invention further provides the functional muscarinic M1/M4 receptor agonist or a pharmaceutically acceptable salt thereof, in particular sabcomeline or a pharmaceutically acceptable salt thereof for use for simultaneous therapeutic administration with at least one atypical antipsychotic agent in the treatment of the negative symptoms of schizophrenia.

The invention further provides the use of at least one neuroprotective agent and/or neuroleptic agent (which may be a typical or atypical antipsychotic agent) in the manufacture of a medicament for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at least one neuroprotective agent in the manufacture of a medicament for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at least one neuroleptic agent in the manufacture of a medicament for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at least one typical antipsychotic agent in the manufacture of a medicament for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at least one atypical antipsychotic agent in the manufacture of a medicament for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at one neuroprotective agent and/or neuroleptic agent (which may be a typical or atypical antipsychotic agent) for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at one neuroprotective agent for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at one neuroleptic agent for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at one typical antipsychotic agent for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

The invention further provides the use of at one atypical antipsychotic agent for simultaneous therapeutic administration with the functional muscarinic M1/M4 receptor agonist or a pharmaceutically acceptable salt thereof, in particular sabcomeline or a pharmaceutically acceptable salt thereof in the treatment of the negative symptoms of schizophrenia.

In further aspects, the invention provides
a method of treatment of the negative symptoms of schizophrenia by simultaneous therapeutic administration of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent), the use of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) for the treatment of the negative symptoms of schizophrenia,
the use of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent), in the manufacture of a medicament for the treatment of the negative symptoms of schizophrenia, and
a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) for use in the treatment of the negative symptoms of schizophrenia.
In further aspects, the invention provides
a method of treatment of the negative symptoms of schizophrenia by simultaneous therapeutic administration of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent, a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent,
the use of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent for the treatment of the negative symptoms of schizophrenia,
the use of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent, in the manufacture of a medicament for the treatment of the negative symptoms of schizophrenia, and
a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent for use in the treatment of the negative symptoms of schizophrenia.
In further aspects, the invention provides a method of treatment of the negative symptoms of schizophrenia by simultaneous therapeutic administration of a pharmaceutical composition comprising the functional muscarinic M1/M4 receptor agonist or a pharmaceutically acceptable salt thereof, in particular sabcomeline or a pharmaceutically acceptable salt thereof and at least one neuroleptic agent (which may be a typical or atypical antipsychotic)
a pharmaceutical composition comprising the functional muscarinic M1/M4 receptor agonist or a pharmaceutically acceptable salt thereof, in particular sabcomeline or a pharmaceutically acceptable salt thereof and at least one neuroleptic agent,
the use of a pharmaceutical composition comprising the functional muscarinic M1/M4 receptor agonist or a pharmaceutically acceptable salt thereof, in particular sabcomeline or a pharmaceutically acceptable salt thereof and at least one neuroleptic agent for the treatment of the negative symptoms of schizophrenia,
the use of a pharmaceutical composition comprising the functional muscarinic M1/M4 receptor agonist or a pharmaceutically acceptable salt thereof, in particular sabcomeline or a pharmaceutically acceptable salt thereof and at least one neuroleptic agent, in the manufacture of a medicament for the treatment of the negative symptoms of schizophrenia, and
a pharmaceutical composition comprising the functional muscarinic M1/M4 receptor agonist or a pharmaceutically acceptable salt thereof, in particular sabcomeline or a pharmaceutically acceptable salt thereof and at least one neuroleptic agent for use in the treatment of the negative symptoms of schizophrenia.
In further aspects, the invention provides
a method of treatment of the negative symptoms of schizophrenia by simultaneous therapeutic administration of a pharmaceutical composition comprising the functional muscarinic M1/M4 receptor agonist or a pharmaceutically acceptable salt thereof, in particular sabcomeline or a pharmaceutically acceptable salt thereof and at least one typical antipsychotic agent,
a pharmaceutical composition comprising the functional muscarinic M1/M4 receptor agonist or a pharmaceutically acceptable salt thereof, in particular sabcomeline or a pharmaceutically acceptable salt thereof and at least one typical antipsychotic agent,
the use of a pharmaceutical composition comprising the functional muscarinic M1/M4 receptor agonist or a pharmaceutically acceptable salt thereof, in particular sabcomeline or a pharmaceutically acceptable salt thereof and at least one typical antipsychotic agent for the treatment of the negative symptoms of schizophrenia,
the use of a pharmaceutical composition comprising the functional muscarinic M1/M4 receptor agonist or a pharmaceutically acceptable salt thereof, in particular sabcomeline or a pharmaceutically acceptable salt thereof and at least one typical antipsychotic agent, in the manufacture of a medicament for the treatment of the negative symptoms of schizophrenia, and
a pharmaceutical composition comprising the functional muscarinic M1/M4 receptor agonist or a pharmaceutically acceptable salt thereof, in particular sabcomeline or a pharmaceutically acceptable salt thereof and at least one typical antipsychotic agent for use in the treatment of the negative symptoms of schizophrenia.
In further aspects, the invention provides
a method of treatment of the negative symptoms of schizophrenia by simultaneous therapeutic administration of a pharmaceutical composition comprising the functional muscarinic M1/M4 receptor agonist or a pharmaceutically acceptable salt thereof, in particular sabcomeline or a pharmaceutically acceptable salt thereof and at least one atypical antipsychotic agent,
a pharmaceutical composition comprising the functional muscarinic M1/M4 receptor agonist or a pharmaceutically acceptable salt thereof, in particular sabcomeline or a pharmaceutically acceptable salt thereof and at least one atypical antipsychotic agent,
the use of a pharmaceutical composition comprising the functional muscarinic M1/M4 receptor agonist or a pharmaceutically acceptable salt thereof, in particular sabcomeline or a pharmaceutically acceptable salt thereof and at least one atypical antipsychotic agent for the treatment of the negative symptoms of schizophrenia,
the use of a pharmaceutical composition comprising the functional muscarinic M1/M4 receptor agonist or a pharmaceutically acceptable salt thereof, in particular sabcomeline or a pharmaceutically acceptable salt thereof and at least one atypical antipsychotic agent, in the manufacture of a medicament for the treatment of the negative symptoms of schizophrenia, and
a pharmaceutical composition comprising the functional muscarinic M1/M4 receptor agonist or a pharmaceutically acceptable salt thereof, in particular sabcomeline or a pharmaceutically acceptable salt thereof and at least one atypical antipsychotic agent for use in the treatment of the negative symptoms of schizophrenia.

In a further aspect, the invention provides a kit for use in the treatment of the negative symptoms of schizophrenia comprising a first dosage form comprising the functional muscarinic M1/M4 receptor agonist or a pharmaceutically acceptable salt thereof, in particular sabcomeline or a pharmaceutically acceptable salt thereof and one or more further dosage forms each comprising a neuroprotective agent and/or neuroleptic agent (which may be a typical or atypical antipsychotic agent) for simultaneous therapeutics administration.

In a further aspect, the invention provides a kit for use in the treatment of the negative symptoms of schizophrenia comprising a first dosage form comprising the functional muscarinic M1/M4 receptor agonist or a pharmaceutically acceptable salt thereof, in particular sabcomeline or a pharmaceutically acceptable salt thereof and one or more further dosage forms each comprising a neuroprotective agent for simultaneous therapeutics administration.

In a further aspect, the invention provides a kit for use in the treatment of the negative symptoms of schizophrenia comprising a first dosage form comprising the functional muscarinic M1/M4 receptor agonist or a pharmaceutically acceptable salt thereof, in particular sabcomeline or a pharmaceutically acceptable salt thereof and one or more further dosage forms each comprising a neuroleptic agent for simultaneous therapeutics administration.

In a further aspect, the invention provides a kit for use in the treatment of the negative symptoms of schizophrenia comprising a first dosage form comprising the functional muscarinic M1/M4 receptor agonist or a pharmaceutically acceptable salt thereof, in particular sabcomeline or a pharmaceutically acceptable salt thereof and one or more further dosage forms each comprising a typical antipsychotic agent for simultaneous therapeutics administration.

In a further aspect, the invention provides a kit for use in the treatment of the negative symptoms of schizophrenia comprising a first dosage form comprising the functional muscarinic M1/M4 receptor agonist or a pharmaceutically acceptable salt thereof, in particular sabcomeline or a pharmaceutically acceptable salt thereof and one or more further dosage forms each comprising an atypical antipsychotic agent for simultaneous therapeutics administration.

In all the foregoing aspects the negative symptoms may be primary or secondary negative symptoms. They may thus be affective flattening, alogia, avolition, anhedonia, dysphoric mood, disturbances in sleep pattern, and lack of insight. They may thus also be movement disorders, such as extrapyramidal symptoms, akathisia and tardive dyskinesia, and demoralisation.

It has also been found that the functional muscarinic M1/M4 receptor agonist or a pharmaceutically acceptable salt thereof, in particular sabcomeline or a pharmaceutically acceptable salt thereof may advantageously be administered in combination with at least one anti-schizophrenic agent or drug to provide treatment of both positive and negative symptoms of schizophrenia. The combination of the functional muscarinic M1/M4 receptor agonist or a pharmaceutically acceptable salt thereof, in particular sabcomeline and anti-schizophrenic agent may also be used with at least one neuroprotective agent and/or neuroleptic agent (which may be a typical or atypical antipsychotic) and/or atypical antipsychotic agent and/or atypical antipsychotic agent to provide improved treatment of both the positive and negative symptoms of schizophrenia.

The combination of the functional muscarinic M1/M4 receptor agonist or a pharmaceutically acceptable salt thereof, in particular sabcomeline and anti-schizophrenic agent may in particular be used with at least one neuroprotective agent to provide improved treatment of the negative symptoms of schizophrenia.

A neuroprotective agent may be defined as a compound which is intended to help limit the damage suffered by a nerve or neural tissue such as, for example, spinal cord, brain or nerve, when the blood supply is cut off or there is a traumatic injury. It is envisaged that psychotic disorders or diseases may be due in part to the abnormalities of neurons or synaptic function or architecture such as to cause a breakdown of neural integrity. It is believed that neuroprotective agents help prevent or stop the breakdown of neurons and neural integrity. Administering a neuroprotective agent alters the underlying pathology affecting integrity of neural function.

Neuroprotective agents include, but are not limited to, some types of antioxidants, anti-inflammatories and antimanic and mood stabiliser drugs such as lithium. Preferably, the neuroprotective agent is selected from the group consisting of anti-oxidants, for example Vitamin E, eicosapentaenoic acid (EPA), docosahexaenoic acid (DHA), and anti-inflammatories, such as non-steroidal anti-inflammatories, cyclo-oxygenase-2 (cox-2) inhibitors, and statins.

When the chosen neuroprotective agent is also an antipsychotic agent (typical or atypical), it is believed that the clinical utility of the combination of the functional muscarinic M1/M4 receptor agonist or a pharmaceutically acceptable salt thereof, in particular sabcomeline and antipsychotic agent may vary between different members of the atypical antipsychotic agent drug class, depending on their different affinities for various sub-types of neurochemical receptors.

For example, in addition to their affinities for dopamine and serotonin receptors, members of the atypical antipsychotic agent class may vary in their affinity for muscarinic and histamine receptor sub-types.

The activity of atypical antipsychotic agents at muscarinic receptor subtypes are such that properties of negligible affinity, weak agonist activity and weak antagonist activity have been reported amongst the various members of the atypical antipsychotic agent drug class.

As an example, the M1/M4 receptor agonist or a pharmaceutically acceptable salt thereof properties of the functional muscarinic M1/M4 receptor agonist or a pharmaceutically acceptable salt thereof, in particular sabcomeline may enhance functional cholinergic activity and, when administered in combination, provide benefit by:
i) enhancing functional cholinergic activity in combination with an atypical antipsychotic agent that itself has little or no affinity for muscarinic receptors (e.g. risperidone);
ii) ii) providing additive functional cholinergic activity in combination with an atypical antipsychotic agent drug that has weak muscarinic receptor agonist or a pharmaceutically acceptable salt thereof effects (e.g. clozapine or N-desmethylclozapine); and/or
iii) competing for muscarinic receptors and thereby reducing the anticholinergic functional effects of an atypical antipsychotic agent drug that possesses muscarinic receptor antagonist properties (e.g. olanzepine).

As well as muscarinic and histaminergic receptors there are other mechanisms that may have beneficial or adverse effects on cognition. For instance drugs with 5-HT6 receptor antagonist and adrenergic α2 receptor antagonist properties may also be of benefit. Some atypical antipsychotics also have these benefits.

A particular example of a neuroprotective agent useful in the invention, its typical route of administration and dosage ranges that are preferred is lithium, trade name Priadel, oral tablet or liquid, 100-1000 gms titrated to plasma level.

The combination of the functional muscarinic M1/M4 receptor agonist or a pharmaceutically acceptable salt thereof, in particular sabcomeline and anti-schizophrenic agent may in particular be used with at least one neuroleptic agent (which may be a typical or atypical antipsychotic) to provide improved treatment of the negative symptoms of schizophrenia.

The term neuroleptic refers to drugs which have the effect on cognition and behaviour of antipsychotic agent drugs that reduce confusion, delusions, hallucinations, and psychomotor agitation in patients with psychoses. Also known as major tranquilizers and antipsychotic drugs, neuroleptic agents include, but are not limited to:

typical antipsychotic drugs, including phenothiazines, further divided into the aliphatics, piperidines, and piperazines, thioxanthenes (e.g., droperidol), butyrophenones (e.g., haloperidol), dibenzoxazepines (e.g., loxapine), dihydroindolones (e.g., molindone), diphenylbutylpiperidines (e.g., pimozide), and
a typical antipsychotic drugs, including benzisoxazoles (e.g., risperidone), olanzapine, quetiapine, osanetant and ziprasidone.

A particular example of a neuroprotective agent useful in the invention, its typical route of administration and dosage ranges that are preferred is olanzapine, trade name Zyprexa, oral tablet, 5 to 20 mg.

Particularly preferred neuroleptic agent for use in the invention are olanzapine, risperidone, quetiapine, aripiprazole, haloperidol, clozapine, ziprasidone and osanetant.

For example, the functional muscarinic M1/M4 receptor agonist or a pharmaceutically acceptable salt thereof, in particular sabcomeline or a pharmaceutically acceptable salt thereof, neuroprotective agent and/or neuroleptic agent (which may be a typical or atypical antipsychotic agent) may be administered, by weight, in the ranges 10 μg-200 μg, 0.0-5 μg, 0.0-5 μg and 0.0-5 μg respectively. Such dosages are typically given once every 12 hours for 3 weeks before being re-assessed by a physician. Preferably, the functional muscarinic M1/M4 receptor agonist or a pharmaceutically acceptable salt thereof, in particular sabcomeline or a pharmaceutically acceptable salt thereof is administered in the range between 20 μg-100 μg. More preferably, the functional muscarinic M1/M4 receptor agonist or a pharmaceutically acceptable salt thereof, in particular sabcomeline or a pharmaceutically acceptable salt thereof is administered in the range between 25 μg-50 μg.

The combination therapies of the invention are preferably administered adjunctively. By adjunctive administration is meant the concurrent or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration.

Any and all treatment regimes in which a patient receives separate but concurrent or overlapping therapeutic administration of the functional muscarinic M1/M4 receptor agonist or a pharmaceutically acceptable salt thereof, in particular sabcomeline or a pharmaceutically acceptable salt thereof and at least one neuroprotective and/or neuroleptic agent (which may be a typical or atypical antipsychotic agent) are within the scope of the current invention. In one embodiment of adjunctive therapeutic administration as described herein, a patient is typically stabilised on a therapeutic administration of one or more of the components for a period of time and then receives administration of another component.

Within the scope of this invention, it is preferred that the functional muscarinic M1/M4 receptor agonist or a pharmaceutically acceptable salt thereof, in particular sabcomeline or a pharmaceutically acceptable salt thereof is administered as adjunctive therapeutic treatment to patients who are receiving administration of at least one

a) neuroprotective agent, and/or
b) neuroleptic agent (which may be a typical or atypical antipsychotic).

However, the scope of the invention also includes the adjunctive therapeutic administration of at least one

a) neuroprotective agent, and/or
b) neuroleptic agent (which may be a typical or atypical antipsychotic agent)
to patients who are receiving administration of the functional muscarinic M1/M4 receptor agonist or a pharmaceutically acceptable salt thereof, in particular sabcomeline or a pharmaceutically acceptable salt thereof.

The combination therapies of the invention may also be administered simultaneously. By simultaneous administration is meant a treatment regime wherein the individual components are administered together, either in the form of a single pharmaceutical composition or device comprising or containing two or more components, or as separate compositions or devices, each comprising one of the components, administered simultaneously. Such combinations of the separate individual components for simultaneous combination may be provided in the form of a kit.

The treatment of the negative symptoms of schizophrenia with the functional muscarinic M1/M4 receptor agonist or a pharmaceutically acceptable salt thereof, in particular sabcomeline or with the functional muscarinic M1/M4 receptor agonist or a pharmaceutically acceptable salt thereof, in particular sabcomeline and at least one neuroprotective agent and/or neuroleptic agent (which may be a typical or atypical antipsychotic agent) as defined in the present invention may occur in addition to further drug therapies.

The functional muscarinic M1/M4 receptor agonist or a pharmaceutically acceptable salt thereof, in particular sabcomeline may also be used in various combined ways with at least one neuroprotective agent and/or neuroleptic agent (which may be a typical or atypical antipsychotic agent).

In a further aspect, the invention provides a pharmaceutical composition comprising
a) the functional muscarinic M1/M4 receptor agonist or a pharmaceutically acceptable salt thereof, in particular sabcomeline or a pharmaceutically acceptable salt thereof and
b) one or more pharmaceutically acceptable excipients.

The composition may also comprise one neuroprotective and/or neuroleptic agent (which may be a typical or atypical antipsychotic agent).

The invention thus provides a pharmaceutical composition comprising the functional muscarinic M1/M4 receptor agonist or a pharmaceutically acceptable salt thereof, in particular sabcomeline or a pharmaceutically acceptable salt thereof and at least one neuroprotective and/or neuroleptic agent (which may be a typical or atypical antipsychotic agent) and one or more pharmaceutically acceptable excipients.

The invention thus provides a pharmaceutical composition comprising the functional muscarinic M1/M4 receptor agonist or a pharmaceutically acceptable salt thereof, in particular sabcomeline or a pharmaceutically acceptable salt thereof and at least one neuroprotective agent and one or more pharmaceutically acceptable excipients.

The invention thus provides a pharmaceutical composition comprising the functional muscarinic M1/M4 receptor agonist or a pharmaceutically acceptable salt thereof, in particular sabcomeline or a pharmaceutically acceptable salt thereof and at least one neuroleptic agent and one or more pharmaceutically acceptable excipients.

The invention thus provides a pharmaceutical composition comprising the functional muscarinic M1/M4 receptor agonist or a pharmaceutically acceptable salt thereof, in particular sabcomeline or a pharmaceutically acceptable salt thereof and at least one typical antipsychotic agent and one or more pharmaceutically acceptable excipients.

The invention thus provides a pharmaceutical composition comprising the functional muscarinic M1/M4 receptor agonist or a pharmaceutically acceptable salt thereof, in particular sabcomeline or a pharmaceutically acceptable salt thereof and at least one atypical antipsychotic agent and one or more pharmaceutically acceptable excipients.

For example, the pharmaceutical composition may comprise the functional muscarinic M1/M4 receptor agonist or a pharmaceutically acceptable salt thereof, in particular sabcomeline, neuroprotective agent and/or neuroleptic agent (which may be a typical or atypical antipsychotic agent), by weight, in the ranges 10 μg-200 μg, 0.0-5 μg, 0.0-5 μg and 0.0-5 μg respectively.

For example, the pharmaceutical composition may comprise the functional muscarinic M1/M4 receptor agonist or a pharmaceutically acceptable salt thereof, in particular sabcomeline and neuroprotective agent, by weight, in the ranges 10 μg-200 μg and 0.0-5 μg respectively.

For example, the pharmaceutical composition may comprise the functional muscarinic M1/M4 receptor agonist or a pharmaceutically acceptable salt thereof, in particular sabcomeline and neuroleptic agent, by weight, in the ranges 10 μg-200 μg and 0.0-5 μg respectively.

For example, the pharmaceutical composition may comprise the functional muscarinic M1/M4 receptor agonist or a pharmaceutically acceptable salt thereof, in particular sabcomeline and typical antipsychotic agent, by weight, in the ranges 10 μg-200 μg and 0.0-5 μg respectively.

For example, the pharmaceutical composition may comprise the functional muscarinic M1/M4 receptor agonist or a pharmaceutically acceptable salt thereof, in particular sabcomeline and atypical antipsychotic agent, by weight, in the ranges 10 μg-200 μg and 0.0-5 μg respectively.

Such dosages are typically given once every 12 hours for 3 weeks before being re-assessed by a physician. Preferably, the functional muscarinic M1/M4 receptor agonist or a pharmaceutically acceptable salt thereof, in particular sabcomeline is present in the range between 20 μg-100 μg. More preferably, the functional muscarinic M1/M4 receptor agonist or a pharmaceutically acceptable salt thereof, in particular sabcomeline is present in the range between 25 μg-50 μg.

The choice of the most appropriate pharmaceutical compositions is within the skill of the art.

Suitable formulations include, but are not limited to tablets, capsules, powders, granules, lozenges, suppositories, reconstitutable powders, or liquid preparations such as oral or sterile parenteral solutions or suspensions.

It is preferred that the compositions used in the invention, are in the form of a unit dose.

Solid unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling, tabletting or the like. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations for use in the invention may be in the form of, for example, emulsions, syrups, suspensions or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, or hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration (for example intravenous, intravascular or subcutaneous administration) of compositions for use in the invention, fluid unit dosage forms are prepared utilizing the component or the combination of the components and a sterile vehicle, and, depending on the concentration used, can be either suspended or dissolved in the vehicle.

In preparing solutions the monotherapy component, the components of the combination therapy or the combination of the components can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum.

Parenteral suspensions are prepared in substantially the same manner, except that the component is suspended in the vehicle instead of being dissolved, and sterilization cannot be accomplished by filtration.

The monotherapy component, the components of the combination therapy, or the combination of the components can be sterilized by exposure to ethylene oxide before suspending in the sterile vehicle. Advantageously, a surfactant or wetting agent is included in the composition to facilitate uniform distribution of the component or the combination of the components.

Alternatively, the components may be prepared in solid form which melts on contact with the tongue of the patient, for example in the form of orally disintegrating tablets sold under the trade name ZYDIS®.

The compositions for use in the invention may be prepared as depot preparations. Such long acting formulations may be administered by implantation (for example subcutaneously or intramuscularly) or by intramuscular injection. Thus, for example, the monotherapy component, the components of the combination therapy or the combination of the components of the invention may be formulated with suitable polymeric or hydrophobic materials (for example as an emulsion in an acceptable oil) or ion exchange resins, or as sparingly soluble derivatives, for example, as a sparingly soluble salt.

The compositions for use in the invention may contain from 0.1% to 99% by weight, preferably from 10-60% by weight, of the active material, depending on the method of administration.

The unit dose of the functional muscarinic M1/M4 receptor agonist or a pharmaceutically acceptable salt thereof, in particular sabcomeline or a pharmaceutically acceptable salt thereof component in compositions according to the invention is in the range of 10 μg-200 μg. Preferably, the dose is between 20 μg-100 μg. More preferably, the dose is between 25 μg-50 μg. The dose may be administered as a single dose or twice daily. Ideally, the functional muscarinic M1/M4 receptor agonist or a pharmaceutically acceptable salt thereof, in particular sabcomeline or a pharmaceutically acceptable salt thereof is administered at a dose of 25 μg twice daily.

For the combination therapies, the daily and unit doses of the neuroprotective agent and/or neuroleptic agent (which may be a typical or atypical antipsychotic agent) will depend upon which neuroprotective agent and/or neuroleptic agent that is employed, but may typically be the recommended or approved dosage for the specific neuroprotective agent and/or neuroleptic agent when administered as monotherapy.

In a preferred aspect of the invention, adjunctive administration of the functional muscarinic M1/M4 receptor agonist or a pharmaceutically acceptable salt thereof, in particular sabcomeline or a pharmaceutically acceptable salt thereof may permit lower doses of the neuroprotective agent and/or neuroleptic agent (which may be a typical or atypical antipsychotic agent) than those normally recommended when the neuroprotective and/or neuroleptic agent is prescribed as monotherapy.

### EXAMPLE 1

An example of a method of preparation of the functional muscarinic M1/M4 receptor agonist or a pharmaceutically acceptable salt thereof, in particular sabcomeline is as follows: to a stirred solution of potassium tert.-butoxide (94.1 g; 0.84 mol) in tetrahydrofuran (250 ml) under nitrogen is added a solution of 3-(cyanomethyl)quinucidine (60 g; 0.4 mol) in tetrahydrofuran (150 ml) during a period of 10 mins. The reaction is stirred for 10 minutes then cooled to 0° C. Isoamyl nitrite (51.5 g 0.44 mol) is added at a rate such that the internal temperature does not exceed 25° C. The reaction is stirred for 20 minutes then diluted with dimethylsulphoxide (500 ml).

Methyl tosylate (134 g; 0.72 mol) is added as a solution in dimethylsulphoxide (100 ml) at a rate such that the temperature does not exceed 35° C. After a further 20 minutes aqueous potassium carbonate (ca 5 wt % 500 ml) is added and the reaction extracted with ethyl acetate (5×200 ml). The ethyl acetate extract is washed with 5 wt % aqueous potassium carbonate (4×250 ml), then saturated potassium carbonate (50 ml). The combined aqueous layers are re-extracted with ethyl acetate (500 ml) which is washed as above. The combined organic extracts are dried over anhydrous potassium carbonate (200 g) and concentrated in vacuo to give a brown oil containing ca. 80 wt % 3-[(cyano)(methoxyimino)-methyl]quinuclidine as a 4:1 mixture of Z:E isomers, (47.4 g; 0.245 mol; 61%).

### EXAMPLE 2

The following patient study was a small Phase IIa, proof of concept, 51-day, multicentre, double-blind, placebo-controlled, rising dose parallel study of the efficacy and tolerability of sabcomeline in patients with acute exacerbation of chronic schizophrenia. A total of twenty eight patients, nineteen received sabcomeline and nine patients received placebo. Daily doses of the functional muscarinic M1/M4 receptor agonist or a pharmaceutically acceptable salt thereof, in particular sabcomeline were titrated from 50 μg daily through 100 μg to 150 μg daily over nine days.

The Primary Objective of the study was:
To assess the efficacy of sabcomeline in patients with schizophrenia (includes: effects on positive and negative symptoms of schizophrenia and general psychopathology)
   The Secondary Objectives of the study were:
To assess the effects of the functional muscarinic M1/M4 receptor agonist or a pharmaceutically acceptable salt thereof, in particular sabcomeline on neurocognitive function in patients with schizophrenia
To study the safety and tolerability of the functional muscarinic M1/M4 receptor agonist or a pharmaceutically acceptable salt thereof, in particular sabcomeline in the treatment of patients with schizophrenia.
   The results of the study showed the following:
   No effect on PANSS positive symptoms: delusions, hallucinations
   A trend in the PANSS negative subscale
   Benefit seen:
PANSS Negative—blunted affect, emotional withdrawal, difficulty in abstract thinking, lack of spontaneity, stereotypical thinking
   No benefit:
PANSS Negative—poor rapport, passive/apathetic social withdrawal

This study was not powered to show statistical significance but rather to generate hypotheses about the potential efficacy of the functional muscarinic M1/M4 receptor agonist or a pharmaceutically acceptable salt thereof, in particular sabcomeline in the treatment of the positive, negative and cognitive symptoms of schizophrenia. Analysis of the data for 8 severe patients and 4 placebo set out below demonstrated that the functional muscarinic M1/M4 receptor agonist or a pharmaceutically acceptable salt thereof, in particular sabcomeline had benefit in the treatment of primary and secondary negative symptoms.

TABLE
Positive and Negative syndrome Scale (PANSS) Item analysis on each
patient with at least moderate severity (≧4) at baseline

| | Sabcomeline | | Placebo | |
|---|---|---|---|---|
| | Total N = 8 | | Total N = 4 | |
| PANSS | Baseline | Endpoint | Baseline | Endpoint |
| Positive Items | | | | |
| Delusions | 8 | 2/8 | 4 | 3/4 |
| Hallucinations | 4 | 2/4 | 4 | 4/4 |
| Negative Items | | | | |
| Blunted Affect | 8 | 7/8 | 0 | 2* |
| Emotional Withdrawal | 7 | 7/7 | 1 | 1/1 |
| Poor rapport | 4 | 3/4 | 2 | 2/2 |
| Passive/Apathetic | 6 | 2/6 | 3 | 0/3 |
| Social Withdrawal | | | | |
| Difficulty in | 6 | 2/6 | 2 | 1/2 |
| abstract thinking | | | | |
| Lack of spontaneity | 6 | 4/6 | 0 | 0 |
| Stereotypical thinking | 3 | 3/3 | 2 | 2/2 |

*indicates worsening of symptoms

The safety and tolerability data for the functional muscarinic M1/M4 receptor agonist or a pharmaceutically acceptable salt thereof, in particular sabcomeline in this study are consistent with observations from clinical studies in other indications (see Example 3) which reveal a generally well-tolerated and safe compound.

### EXAMPLE 3

The functional muscarinic M1/M4 receptor agonist or a pharmaceutically acceptable salt thereof, in particular sabcomeline 50 fÊg daily, or 25 fÊg twice daily, has also be evaluated in two 24-week placebo-controlled trials that included 880 patients with Alzheimer' disease. The functional muscarinic M1/M4 receptor agonist or a pharmaceutically acceptable salt thereof, in particular sabcomeline was safe and well-tolerated across the dose range examined.

1.-56. (canceled)

57. A method of treatment of the negative symptoms of schizophrenia by administration of a functional muscarinic M1/M4 receptor agonist or a pharmaceutically acceptable salt thereof.

58. The method as claimed in claim 57, wherein the negative symptoms are affective flattening, alogia, avolition, anhedonia, dysphoric mood, disturbances in sleep pattern, lack of insight, extrapyramidal symptoms, akathisia and tardive dyskinesia, and/or demoralisation.

59. The method as claimed in claim 57, wherein the functional muscarinic M1/M4 receptor agonist is other than sabcomeline.

60. The method as claimed in claim 57, wherein said treatment comprises administering the functional muscarinic M1/M4 receptor agonist or a pharmaceutically acceptable salt thereof at a dose of between 10 μg-200 μg.

61. The method as claimed in claim 57, wherein said treatment comprises administering the functional muscarinic M1/M4 receptor agonist or a pharmaceutically acceptable salt thereof at a dose of between 25 μg-50 μg.

62. The method as claimed in claim 57 wherein said treatment comprises administering the functional muscarinic M1/M4 receptor agonist or a pharmaceutically acceptable salt thereof as a single dose or twice daily.

63. A method of treatment of the negative symptoms of schizophrenia by adjunctive therapeutic administration of a functional muscarinic M1/M4 receptor agonist or a pharmaceutically acceptable salt thereof to a patient receiving therapeutic administration of at least one neuroprotective agent and/or neuroleptic agent.

64. A method of treatment of the negative symptoms of schizophrenia by adjunctive therapeutic administration of a functional muscarinic M1/M4 receptor agonist or a pharmaceutically acceptable salt thereof to a patient receiving therapeutic administration of at least one typical antipsychotic agent.

65. A method of treatment of the negative symptoms of schizophrenia by adjunctive therapeutic administration of a functional muscarinic M1/M4 receptor agonist or a pharmaceutically acceptable salt thereof to a patient receiving therapeutic administration of at least one atypical antipsychotic agent.

66. The method as claimed in claim 63 wherein the neuroprotective agent is selected from the group consisting of anti-oxidants, eicosapentaenoic acid (EPA), docosahexaenoic acid (DHA), anti-inflammatories, cyclo-oxygenase-2 (cox-2) inhibitors, statins and lithium.

67. The method ask claimed in claim 63, wherein the neuroleptic agent is selected from the group consisting of the following seven classes of drugs: phenothiazines, thioxanthenes, butyrophenones, dibenzoxazepines, dihydroindolones, diphenylbutyl-piperidines and benzisoxazoles.

68. The method as claimed in claim 63, wherein the said treatment comprises administering the functional muscarinic M1/M4 receptor agonist or a pharmaceutically acceptable salt thereof and neuroprotective agent, and/or neuroleptic agent, by percentage weight, in the ranges 1-100%, 0.0-99% and 0.0-99% respectively.

69. The method as claimed in claim 64, wherein the said treatment comprises administering the functional muscarinic M1/M4 receptor agonist or a pharmaceutically acceptable salt thereof and typical antipsychotic by percentage weight, in the ranges 1-100% and 0.0-99% respectively.

70. The method as claimed in claim 65, wherein the said treatment comprises administering the functional muscarinic M1/M4 receptor agonist or a pharmaceutically acceptable salt thereof and atypical antipsychotic by percentage weight, in the ranges 1-100% and 0.0-99% respectively.

71. The method as claimed in claim 63, wherein the said treatment comprises administering the functional muscarinic M1/M4 receptor agonist or a pharmaceutically acceptable salt thereof, neuroprotective agent and/or neuroleptic agent, by weight, in the ranges 10 μg-200 μg, 0.0-5 μg and 0.0-5 μg respectively.

72. The method as claimed in claim 64, wherein the said treatment comprises administering the functional muscarinic M1/M4 receptor agonist or a pharmaceutically acceptable salt thereof and a typical antipsychotic agent, by weight, in the ranges 10 μg-200 μg and 0.0-5 μg respectively.

73. The method as claimed in claim 65, wherein the said treatment comprises administering the functional muscarinic M1/M4 receptor agonist or a pharmaceutically acceptable salt thereof and an atypical antipsychotic agent, by weight, in the ranges 10 μg-200 μg and 0.0-5 μg respectively.

74. A kit for use in the treatment of the negative symptoms of schizophrenia comprising a first dosage form comprising the functional muscarinic M1/M4 receptor agonist or a pharmaceutically acceptable salt thereof, and at least one further dosage form comprising a neuroprotective agent and/or neuroleptic agent for simultaneous therapeutic administration.

75. A kit for use in the treatment of the negative symptoms of schizophrenia comprising a first dosage form comprising the functional muscarinic M1/M4 receptor agonist or a pharmaceutically acceptable salt thereof, and at least one further dosage form comprising a typical antipsychotic agent for simultaneous therapeutic administration.

76. A kit for use in the treatment of the negative symptoms of schizophrenia comprising a first dosage form comprising the functional muscarinic M1/M4 receptor agonist or a pharmaceutically acceptable salt thereof, and at least one further dosage form comprising an atypical antipsychotic agent for simultaneous therapeutic administration.

77. The kit as claimed in claim 74, wherein the neuroprotective agent is selected from the group consisting of anti-oxidants, eicosapentaenoic acid (EPA), 10-docosahexenoic acid (DHA), anti-inflammatories, cyclo-oxygenase-2 (cox-2) inhibitors, statins and lithium.

78. The kit as claimed in claim 74, wherein the neuroleptic agent is selected from the group consisting of the following seven classes of drugs: phenothiazines, thioxanthenes, butyrophenones, dibenzoxazepines, dihydroindolones, diphenyl-butylpiperidines and benzisoxazoles.

79. The kit as claimed in claim 74, wherein said kit comprises the functional muscarinic M1/M4 receptor agonist or a pharmaceutically acceptable salt thereof in a unit dose of between 10 μg-200 μg.
